(19) United States
(12) Patent Application Publication
(10) Pub. No.: US 2018/0153728 A1
(43) Pub. Date: Jun. 7, 2018

(54) A NASAL SPLINT

(71) Applicant: TUAN ANH LE PTY LTD, Lakemba, New South Wales (AU)

(72) Inventor: Andrew LE, Lakemba, New South Wales (AU)

(73) Assignee: TUAN ANH LE PTY LTD, Lakemba, New South Wales (AU)

(21) Appl. No.: 15/737,569

(22) PCT Filed: Jun. 15, 2016
(86) PCT No.: PCT/AU2016/050492

§ 371 Date: Dec. 18, 2017

(30) Foreign Application Priority Data
Jun. 22, 2015 (AU) 2015902389

(51) Int. Cl. A61F 5/08

(52) U.S. Cl. CPC A61F 5/08

(57) ABSTRACT

A transparent nasal splint to be worn externally after a trauma, nasal surgery or nasal injection. The nasal splint has two nasal wall panels bridged together at a spine having an inverted-u shape cross section. The nasal wall panels are inwardly biased to apply compression and to grip onto the nose. The splint is made of semi-rigid material having suitable resiliency to allow moving of the nasal wall panels in an outward direction and to provide for the inward bias of the nasal panels when the splint is in use. The splint may have a nasal spine made of springs(s) or hinge(s) having suitable resiliency to allow moving of the nasal wall panels in an outward direction and to provide for the inward bias of the nasal panels when in use. The splint is also made of suitable material and design which render it being inconspicuous or not visually obvious.

### FIELD OF THE INVENTION

The present invention relates to a nasal splint. More particularly, the invention relates to a nasal splint worn externally by a patient after trauma, surgery or a procedure on their nose to improve the results or appearance of their nose.

### BACKGROUND

Rhinoplasty is surgery where the patient's nose is corrected, reconstructed or cosmetically enhanced either as a result of trauma or an elective procedure. Non-surgical rhinoplasty is a procedure where an injectable filler substance is used to cosmetically enhance the shape of the patient's nose without an invasive surgery. “Natural rhinoplasty” is an attempt to reshape the nose without any surgery or needle.

After injectable rhinoplasty, it is common to have mild to moderate swelling with occasional bleeding and bruising under the skin. It is also common that the filler is not injected into the intended position or injected asymmetrically. There is a desire of a surgeon and also patient to wear a nasal splint which can help to reduce swelling, bruising, bleeding at the same time correction the asymmetry.

There are people who are born with asymmetrical nose, flat nose, large nose etc. There is desire among some of these people to correct or improve the appearance of their noses using natural method or technology, without going through surgery or injection.

After going through rhinoplasty surgery, it is common for the patient to wear a splint or a cast to protect the nose from external pressure that might distort the end result of the rhinoplasty procedure. The splint helps to reduce the post-operative swelling, bruising and bleeding as well as encourages the skin to re-attach to the nasal cartilage as the swelling goes down.

The prior art nasal splints are typically made of aluminium, thermal plastic or plaster and have hard and sharp edges that pressure the sensitive skin around the nose. This causes damage to the skin and is often painful for the patient. In addition, they are rigid and become loose when the post-operative swelling starts going down. It is common for the surgeon to make several new splints to accommodate the changing nose and to continue to provide the necessary support during the first week or two after the procedure.

The prior art splints being worn by normal people to enhance the appearance of their noses can also be made of plastic material. One prior art nasal splint is a plastic clip which pinch the nostrils from the outside. They are visually obvious and conspicuous so they usually can be used only indoors. They also obstruct the breathing and only can be worn for a short period of time.

Furthermore, the prior art splints obscure the surgeon's vision to inspect post-operative swelling, bruising, inflammation, ulceration etc. and make it difficult to assess the results of the procedure. The prior art splints also require external bandages and tapping, rendering them bulky, uncomfortable to wear, very conspicuous, visually obvious. Therefore, the prior art splints result in cosmetic unacceptance and are unwelcome by many patients, which reduce compliance and increase postoperative complications, and compromise the final outcome.

Any discussion of the background art above or throughout the specification should in no way be considered as an admission that such art is widely known or forms part of common general knowledge in the field.

### SUMMARY OF THE INVENTION

It is an object of the present invention to overcome or ameliorate at least one of the disadvantages of the prior art, or to provide a useful alternative.

One embodiment provides a nasal splint to be worn externally on a patient's nose comprising: a first nasal wall panel and a second nasal wall panel bridged together at a spine having an inverted-u shape as a cross section, the nasal wall panels being adapted to conform to the nasal pyramid shape, and being inwardly biased to apply compression and grip onto a nose.

In an embodiment, the nasal splint is substantially transparent or translucent. The entire nasal splint or the nasal wall panels and/or the nasal bridge supports and/or the nasal spine can be made of transparent material.

In an embodiment, an upper end of the nasal splint further includes two bilateral and inwardly biased nasal bridge supports, the nasal bridge supports being elongate, arched and adapted to grip onto the nasal bridge and the eye orbit when in use.

In an embodiment, the nasal wall panels are movable in an outward direction and resiliently returnable to their original position.

In an embodiment, the nasal bridge supports are movable in an outward direction and resiliently returnable to their original position.

In one embodiment, the nasal splint is made of semi-rigid material having suitable resiliency to allow moving of the nasal wall panels in an outward direction and to provide for the inward bias of the nasal wall panels and the nasal bridge supports when the nasal splint is in use.

In one embodiment, the spine is more flexible than the nasal wall panels. In an embodiment, the nasal bridge supports are more flexible than the nasal wall panels. The nasal splint is preferably a unitary structure but can also consist of separate elements. In an embodiment, the nasal splint includes a groove at a junction where the nasal wall panel meets the nasal bridge support. The spine may include an elongate split adapted to decrease the inward bias of the nasal wall panels and/or the nasal bridge supports.

In an embodiment, the spine includes a spring assembly having one or more springs to connect the nasal wall panels to one another. In another embodiment, the spine includes a hinge assembly having one or more hinges to connect the nasal wall panels to one another.

In an embodiment, each nasal wall panel includes a flange opposite to the spine, the flange extending outwardly from the nasal wall panel and being adapted to grip onto the base of the nose where the nose meets the cheek, when in use.

In an embodiment, a side edge opposite to the spine of each nasal wall panel is rounded. In an embodiment, a side edge opposite to the spine of each nasal wall panel can also be angled upwardly from the skin, when the nasal splint is in use. The outer edges of the nasal bridge supports can also be rounded. The outer edges of the nasal bridge supports can also be angled upwardly from the skin, when the nasal splint is in use.

In one embodiment, each nasal wall panel and/or the nasal bridge supports include one or more trim lines for adjusting the shape and/or size of the splint.

In an embodiment, the nasal splint further includes an adhesive on an inner surface of the splint.

In an embodiment, the nasal splint further includes one or more cushions attached to an inner surface of the nasal splint.

In an embodiment, the nasal splint further includes one or more ventilation apertures on the spine and/or the nasal wall panels.

The nasal splint according to the present invention can be worn even by a normal person to enhance or correct the appearance of their nose. It is a transparent or translucent nasal splint with a design to render it nearly invisible and/or inconspicuous. The nasal splint according to the present invention has an ergonomic design to reduce dead space, to increase the grip and to reduce the discomfort for the user. The rolled edges of the nasal splint reduce demarcation on the skin and discomfort for the user. Furthermore, the nasal splint with its unique compression force on the nose and adhesive inner surfaces is water proof. As a summary, the nasal splint according to the present invention can deliver better results than the prior art nasal splints.

### BRIEF DESCRIPTION OF THE DRAWINGS

Embodiments of the invention will now be described, by way of example only, with reference to the accompanying drawings in which:

FIG. 1 shows a perspective view of the nasal splint;

FIG. 2 shows another perspective view of an embodiment of the nasal splint;

FIG. 3 shows a top view of the nasal splint;

FIG. 4 shows a side view of an embodiment of the nasal splint;

FIG. 5 shows a perspective view of an embodiment of the nasal splint;

FIG. 6 shows a perspective view of the nasal splint viewed from under the splint;

FIG. 7 shows a perspective view of an embodiment of the nasal splint having an elongate opening on the nasal spine;

FIG. 8 shows a perspective view of an embodiment of the nasal splint where the nasal spine comprises a spring assembly; and

FIG. 9 shows a perspective view of the nasal splint where the nasal spine comprise a hinge assembly.

### DETAILED DESCRIPTION

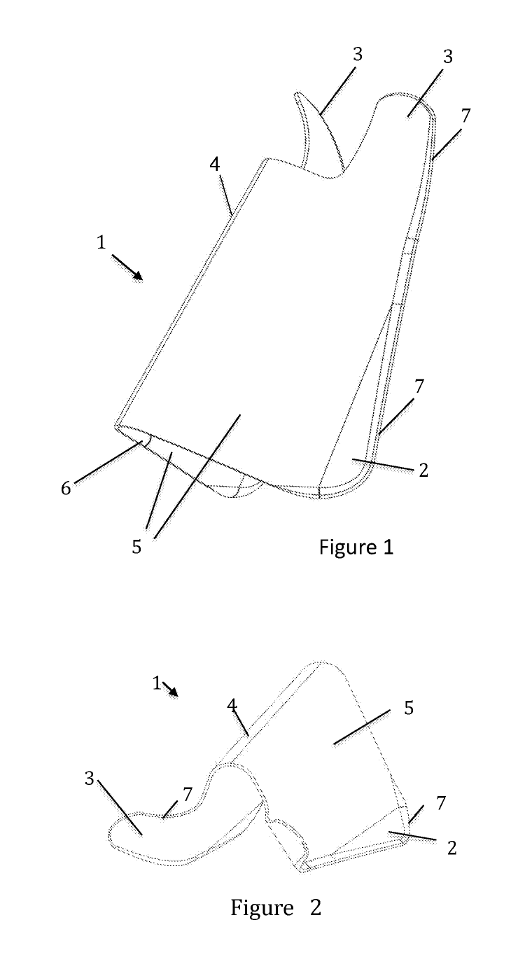

FIG. 1 provides a perspective view of an embodiment of the nasal splint device 1. The nasal splint 1 is made of thin plastics material having a spine (to imitate nasal dorsum) 4 connecting two nasal wall panels 5 to grip onto each side of the patient's nose. The nasal wall panels 5 are inwardly biased so that they apply a gentle force on the patient's nose to maintain the nasal splint in its correct position when it is in use. The nasal wall panels 5 are substantially straight or convex or concave inwardly. In the embodiments of FIGS. 1 to 7 they are either substantially straight or curve outwards and their side edges 7 are preferably rounded as seen in FIGS. 1, 2 and 4. In addition, the embodiment of the nasal splint illustrated in FIGS. 1 to 7 is made out of a single sheet of material so it has a uniform structure.

As seen in the accompanying FIGS. 1-6, the nasal splint 1 also includes two bilateral nasal bridge supports 3. When in use, the nasal bridge supports 3 apply a gentle compression force around the nasal bridge and they also assist in holding the device in place. The bridge supports 3 are symmetrical wing-like limbs that are curved and have a blunt tip. The bridge supports assist in both placing the nasal splint in its correct position and holding it in that position. The bridge supports 3 are adapted to contour the arch from the nasal bridge towards the upper orbit of the eye.

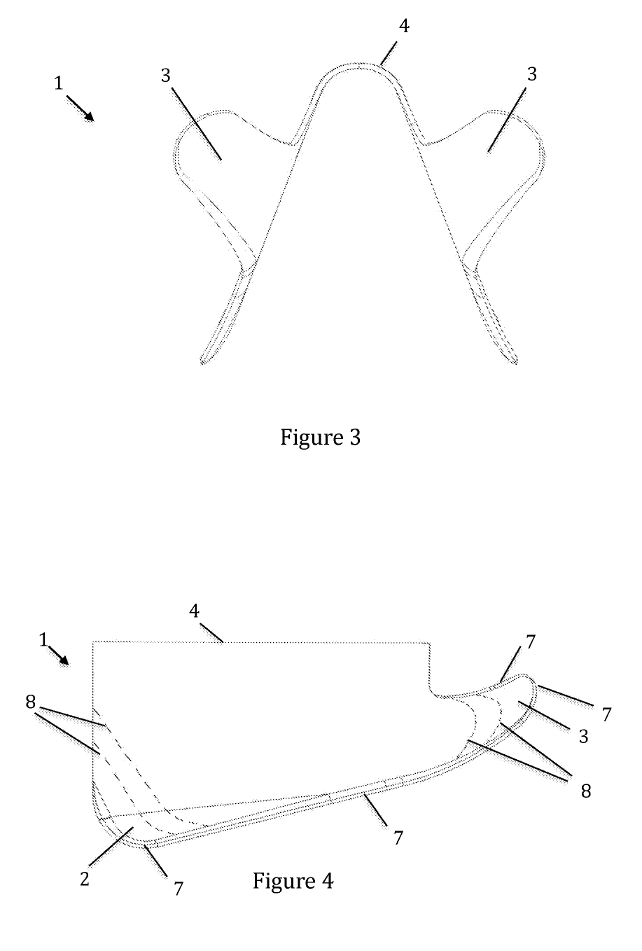

The leaf shaped bridge supports 3 protrude outwardly from the plane of the nasal wall panels 5 as best seen in FIG. 3. As the bridge supports 3 contour the arch from the nose towards the upper part of the eye orbit, eyeglasses can be worn without difficulty. Similarly to the nasal wall panels 5, the bridge supports 3 are inwardly biased so that they apply a gentle compression force to assist keeping the device in its correct position.

As best seen in FIGS. 1, 2 and 4, each nasal wall panel 5 of the splint has a flange 2 that enables the device to follow the contour of the skin at the base of the nose where the nasal bone meets the upper cheek. In addition, the flange has rounded edges 7 so as to maximise the comfort experienced by the patient. The rounded edges 7 of the device extend along the nasal wall panels towards nasal bridge supports 3. In the embodiment of FIGS. 2, 3 and 4, the rounded edges 7 continue all along the side edges of the nasal wall and the nasal bridge supports 3. The edges of the nasal wall panels 5 and the nasal bridge supports 3 may also be angled upwardly from the skin. The rounded edges 7 and any angling of the edges prevent rubbing of the device against the sensitive skin after surgery. In addition, they prevent the edges from causing demarcation and discomfort of the skin.

The nasal splint can be manufactured in one or more different sizes to accommodate the varying sizes of patients' noses. For example, the sizing can be small, medium and large or female and male. Alternatively, sizes small, medium and large can be made for male and female noses separately. Angle 6 between the two side panels 5 is chosen so that it is slightly smaller for a small size splint and larger for a large splint. The angle 6 is larger at the lower end of the nasal splint and smaller at the upper end of the nasal splint (where the nasal bridge supports begin). In addition, the spine, having an inverted-U shaped cross-section, is wider at the lower end of the nasal splint and narrower at the upper end of the nasal splint. The angle (or the size of the inverted-U shape) between the two sides affects the compression force applied by the device on the patient's nose and the skin around the nose. The angle is chosen for each size of the nasal splint so that the nasal wall panels 5 apply a sufficient compression force on the patient's nose to grip onto the nose but so that it does not cause discomfort to the patient. The compression force speeds up the healing process as well as reduces post-operative swelling, bleeding and bruising so it needs to be sufficient but not in excess so as to incur pain to the patient. The compression force is also determined by the elasticity of the material used for the nasal splint.

Nasal bridge supports 3 can be made to be more flexible than the nasal wall panels 5. They can, for example, be made of more flexible material such as rubber or they can be made from the same material as the nasal wall panels 5, but thinner. This will allow the bridge supports 3 to bend according to different shapes of the eye orbit without putting undue pressure on the eye socket.

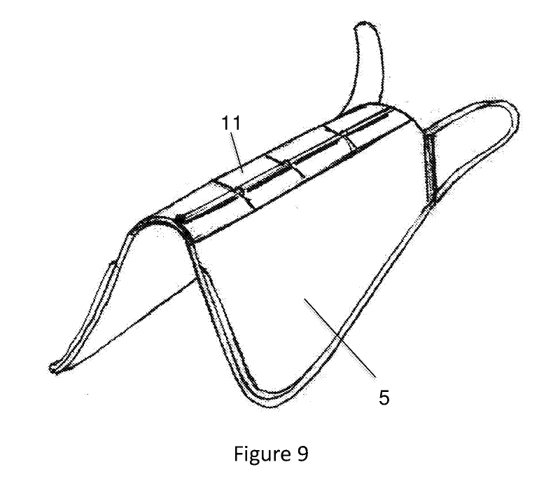

The nasal splint is made of any suitable material, such as a polyester or polyethylene terephthalate, that has suitable properties to be molded into the shape seen in the accompanying figures. The material used has sufficient resiliency to allow bending of the nasal wall panels in an outward direction when applying the nasal splint and to provide for the inward bias of the nasal wall panels and the nasal bridge supports when the nasal splint is in use. Although the embodiment described herein is made of flexible plastics it is intended that other suitable materials such as silicone or synthetics may be used. After the manufacturing process, the nasal splint is semi-rigid but still sufficiently flexible to be able to be applied onto the patient's nose. The spine 4 can also be made of more flexible material than the nasal wall panels such as rubber. Alternatively, it can be made of the same material as nasal wall panels 5 but thinner. This will reduce the compression force that the nasal wall panels exert on the nose to reduce discomfort. The more flexible the nasal spine is, the more variety in nose sizes can be treated i.e. the device can be used for both thinner nose and wider nose without exerting undue pressure on the nose. The embodiments of FIGS. 8 and 9 show a nasal splint having a spine made of different material where the spine includes hinges or springs that are used to determine the inward bias of the nasal wall panels. The different designs of the spine 4 as well as the materials used for the spring or the hinge assembly also affect the compression force of the nasal splint.

In addition, the nasal splint is transparent so that its appearance is cosmetically appealing to the patients. It also allows for the surgeon to examine and monitor the post-operative healing of the nose without removing the device 1.

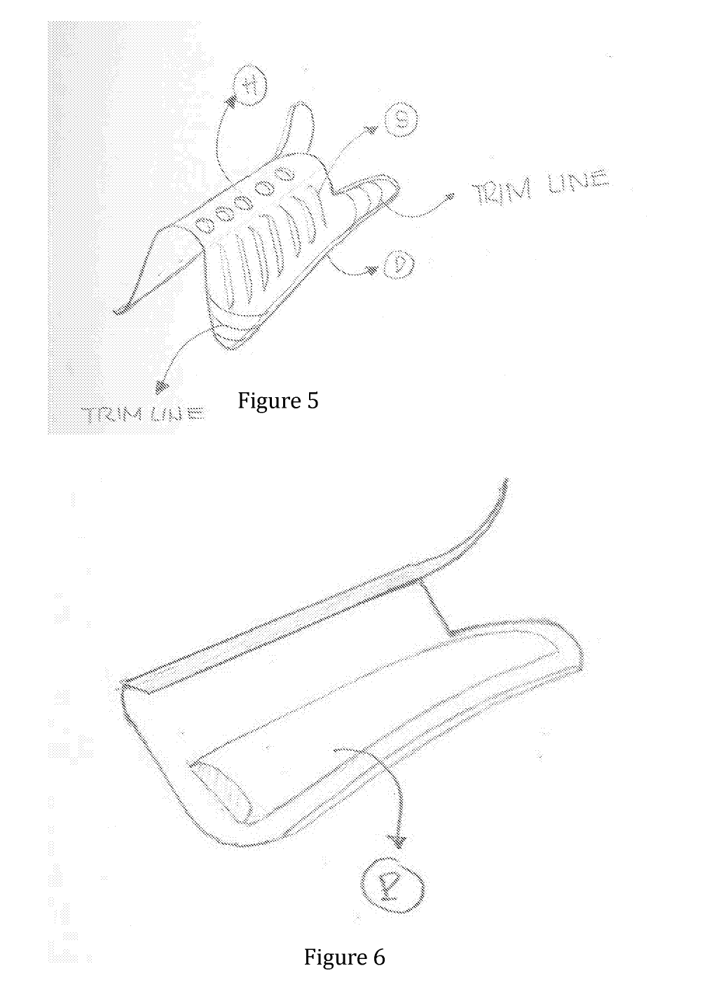

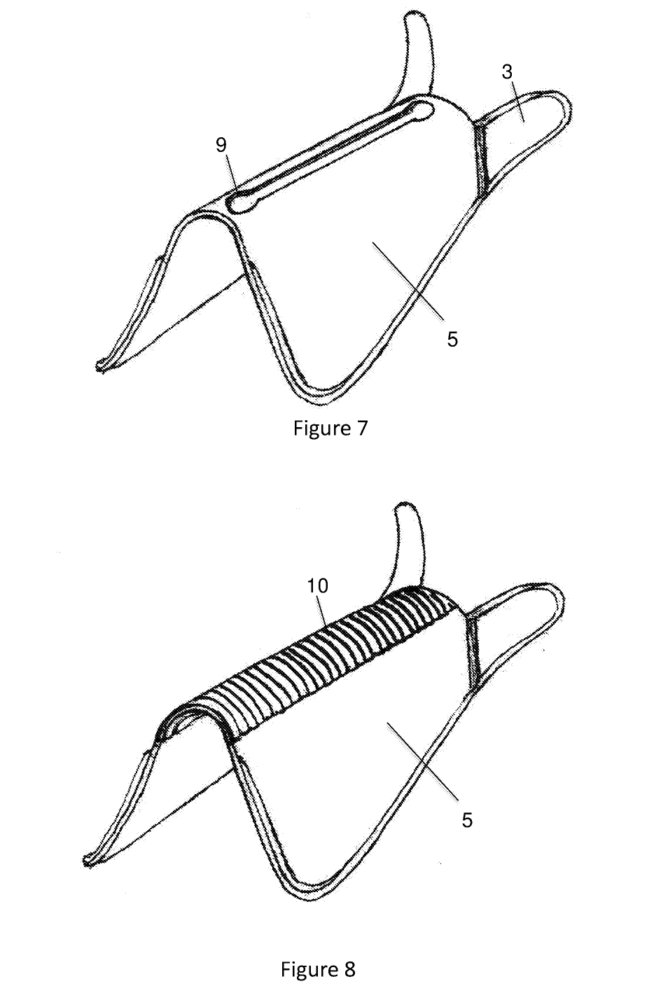

FIGS. 4 and 5 show the nasal splint having one or more trim lines (grooves) 8 that allow trimming of the nasal splint to accommodate the varying sizes of noses. The trim lines 8 are located at the lower end of the nasal walls 5 and preferably also at the upper end of the nasal bridge supports 3. The trim lines are printed, compressed or cut out grooves on the device that allow the surgeon to trim the nasal splint quickly, for example, by using scissors. The trim lines give the surgeon the option to reduce the size of the splint, for example, in such a case where alar compression is not needed. FIG. 5 shows an embodiment of the nasal splint having ventilation holes (H) on the spine and elongate openings (S) on the nasal wall panel 5 to further assist in keeping the skin dry and fresh. The nasal splint is easy to apply on the patient and does not require the use of additional tapes or adhesives. The compression force by the nasal wall panels 5 and the bridge supports 3 is sufficient to hold the device in place over the nose of the patient. However, an adhesive can be used on the nasal wall panels 5 to further assist the device in staying in the correct position. In addition, the nasal splint 1 can have one or more cushions (P), seen in FIG. 6, made of soft material such as silicone to add extra compression and comfort. Furthermore, another cushion such as a dorsal pad can be used to further assist the nasal splint in shaping the nose after an operation, injection or a trauma. A dorsal pad is a foam pad that can be placed under the inverted-U shaped spine 4 to put a slight pressure on the nasal bridge and septum to avoid poly peak deformity.

As seen in FIG. 7, the spine 4 of the nasal splint can also have an elongate opening or split 9. This opening may act as a ventilation hole but it can also be used to reduce the inward bias or the compression force of the nasal wall panels 5. The longer the opening, the less force is exerted by the nasal wall panels 5 onto the patient's nose. The opening has bulbous ends to make the nasal splint more resistant to any strain fractures when the device is in use.

FIG. 8 shows another embodiment of the nasal splint where the spine 4 of the nasal splint comprises a spring assembly 10 having one or more spring elements. The spring assembly 10 may be used to adjust the bias of the nasal wall panels 5. A further embodiment of the nasal splint is shown in FIG. 9 where the spine 4 comprises a spring assembly having one or more hinges which determine compression force of the nasal wall panels.

As seen in FIGS. 7 to 9, the nasal splint includes a groove at the junction where the nasal wall panel meets the nasal bridge support. The groove increases flexibility and reduces the inward bias of the nasal bridge supports.

The nasal splint 1 is light-weight and comfortable to wear. It is also waterproof so it can be worn in the shower. Because it is transparent, it is also cosmetically more appealing and not easily seen by the general public. In addition, the transparency allows for the surgeon to monitor the progress of the healing without removing the device and take action if any complications arise.

The material used for the manufacture must have a sufficient degree of resiliency or springiness to allow pulling the nasal wall panels outwardly when applying the nasal splint without breaking the splint at the spine. The material used may also have different colours and/or different levels of translucency and transparency. Although certain materials and manufacturing processes are discussed above, other suitable materials and methods may be used, as known in the art.

Reference throughout this specification to “one embodiment”, “an embodiment” means that a particular feature, structure or characteristic described in connection with the embodiment is included in at least one embodiment of the present invention. In the claims below and the description herein, any one of the terms comprising, comprised of or which comprises is an open term that means including at least the elements/features that follow, but not excluding others. Thus, the term comprising, when used in the claims, should not be interpreted as being limitative to the means or elements or steps listed thereafter.

Furthermore, while some embodiments described herein include some but not other features included in other embodiments, combination of features of different embodiments are meant to be within the scope of the invention and form different embodiments, as would be understood by those skilled in the art. For example, in the following claims, any of the claimed embodiments can be used in any combination.

While the description includes what are believed to be the preferred embodiments of the invention, those skilled in the art will recognize that other and further modifications may be made thereto without departing from the spirit of the invention, and it is intended to claim all such changes and modifications as falling within the scope of the invention. It will be appreciated by persons skilled in the art that numerous variations and/or modifications may be made to the disclosure as shown in the specific embodiments without departing from the scope of the disclosure as broadly described. The present embodiments are, therefore, to be considered in all respects as illustrative and not restrictive.

1. A nasal splint to be worn externally on a patient's nose comprising:
a first nasal wall panel and a second nasal wall panel bridged together at a spine having an inverted-u shape as a cross section, the nasal wall panels being adapted to conform to the nasal pyramid shape, and being inwardly biased to apply compression and grip onto a nose.

2. The nasal splint according to claim 1, wherein the nasal splint is substantially transparent so as to be visually inconspicuous.

3. The nasal splint according to claim 1, wherein an upper end of the nasal splint further includes two bilateral and inwardly biased nasal bridge supports, the nasal bridge supports being elongate, arched and adapted to grip onto the nasal bridge and the eye orbit when in use.

4. The nasal splint according to claim 1, wherein the nasal wall panels are movable in an outward direction and resiliently returnable to their original position.

5. The nasal splint according to claim 3, wherein the nasal bridge supports are movable in an outward direction and resiliently returnable to their original position.

6. The nasal splint according to claim 3, wherein the nasal splint is made of semi-rigid material having suitable resiliency to allow moving of the nasal wall panels in an outward direction and to provide for the inward bias of the nasal wall panels and the nasal bridge supports when the nasal splint is in use.

7. The nasal splint according to claim 1, wherein the spine is more flexible than the nasal wall panels.

8. The nasal splint according to claim 3, wherein the nasal bridge supports are more flexible than the nasal wall panels.

9. The nasal splint according to claim 1, wherein the nasal splint is a unitary structure.

10. The nasal splint according to claim 3, wherein the nasal splint includes a groove at a junction where the nasal wall panel meets the nasal bridge support.

11. The nasal splint according to claim 1, wherein the spine includes a longitudinal split adapted to decrease the inward bias of the nasal wall panels and/or the nasal bridge supports.

12. The nasal splint according to claim 1, wherein the spine includes a spring assembly having one or more springs to connect the nasal wall panels to one another.

13. The nasal splint according to claim 1, wherein the spine includes a hinge assembly having one or more hinges to connect the nasal wall panels to one another.

14. The nasal splint according to claim 1, wherein each nasal wall panel includes a flange opposite to the spine, the flange extending outwardly from the nasal wall panel and being adapted to grip onto the base of the nose where the nose meets the cheek, when in use.

15. The nasal splint according to claim 1, wherein a side edge opposite to the spine of each nasal wall panel is rounded.

16. The nasal splint according to claim 1, wherein a side edge opposite to the spine of each nasal wall panel is angled upwardly from the skin, when the nasal splint is in use.

17. The nasal splint according to claim 3, wherein outer edges of the nasal bridge supports are rounded.

18. The nasal splint according to claim 3, wherein outer edges of the nasal bridge supports are angled upwardly from the skin, when the nasal splint is in use.

19. The nasal splint according to claim 1, wherein each nasal wall panel includes one or more trim lines for adjusting the shape and/or size of the splint.

20. The nasal splint according to claim 3, wherein the nasal bridge supports include one or more trim lines for adjusting the shape and/or size of the splint.

21. The nasal splint according to claim 1, wherein the nasal splint further includes an adhesive on an inner surface of the splint.

22. The nasal splint according to claim 1, wherein the nasal splint further includes one or more cushions attached to an inner surface of the nasal splint.

23. The nasal splint according to claim 1, wherein the nasal splint further includes one or more ventilation apertures on the spine and/or the nasal wall panels.
